(19) United States
(12) Patent Application Publication
(10) Pub. No.: US 2018/0271587 A1
(43) Pub. Date: Sep. 27, 2018

(54) ENERGY TREATMENT INSTRUMENT

(71) Applicant: OLYMPUS CORPORATION, Tokyo (JP)

(72) Inventors: Akinori KOBAYASHI, Hachioji-shi (JP); Yuki KAWAGUCHI, Koshu-shi (JP)

(73) Assignee: OLYMPUS CORPORATION, Tokyo (JP)

(21) Appl. No.: 15/989,602

(22) Filed: May 25, 2018

Related U.S. Application Data
(63) Continuation of application No. PCT/JP2015/083420, filed on Nov. 27, 2015.

(51) Int. Cl. A61B 18/14; A61B 18/12

(52) U.S. Cl. CPC A61B 18/1445; A61B 2018/00577; A61B 18/1206

(57) ABSTRACT

An energy treatment instrument includes: an end effector provided at a distal end of a support portion; a handle main body supporting the support portion; a movable handle attached to be movable with respect to the handle main body for operating the end effector; and a switch provided on a side surface of the handle main body, and capable of changing an ON/OFF status of an energy output from the end effector when operated in a direction substantially along a longitudinal axis of the support portion.

### CROSS-REFERENCE TO RELATED APPLICATIONS

This application is a Continuation Application of PCT Application No. PCT/JP2015/083420, filed Nov. 27, 2015 the entire contents of all of which are incorporated herein by reference.

### BACKGROUND OF THE INVENTION

### 1. Field of the Invention

The present invention relates to an energy treatment instrument used for surgery.

### 2. Description of the Related Art

Jpn. Pat. Appln. KOKAI Publication No. 2011-143252 discloses an ablation device including an ablation probe extending distally from a distal end of a handle assembly, and a user interface disposed at the handle assembly. The user interface is provided with an intensity controller capable of adjusting power parameters.

Jpn. Pat. Appln. KOKAI Publication No. 2008-212679 discloses a treatment apparatus for operation configured to operate in a plurality of output modes. The treatment apparatus for operation includes an operation switch, and is operated in an output mode selected by the operation switch.

### BRIEF SUMMARY OF THE INVENTION

According to an aspect of the present invention, an energy treatment instrument includes: an end effector provided at a distal end of a support portion; a handle main body supporting the support portion; a movable handle attached to be movable with respect to the handle main body for operating the end effector; and a switch provided on a side surface of the handle main body, and capable of changing an ON/OFF status of an energy output from the end effector when operated in a direction substantially along a longitudinal axis of the support portion.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 a schematic view showing an entire structure of an energy treatment instrument of a first embodiment.

FIG. 2 is a perspective view illustrating an end effector of a handpiece of the energy treatment instrument illustrated in FIG. 1.

FIG. 3 is a cross-sectional view of a vibration generator of the handpiece illustrated in FIG. 2 cut along a plane including a longitudinal axis C of a probe.

FIG. 4 is an enlarged front view showing part of the handpiece illustrated in FIG. 1 held with doctor's right hand.

FIG. 5 is a front view showing a state in which a switch is pushed with a thumb while a movable handle of the handpiece illustrated in FIG. 4 is pulled to a grip portion.

FIG. 6 is a plan view showing a switch and a button viewed from the opposite surface (top surface) direction by cutting away part of the handle main body of the handpiece illustrated in FIG. 4.

FIG. 7 is a plan view showing a state in which the switch of the handpiece illustrated in FIG. 6 is pushed with the thumb of the right hand.

FIG. 8 is a front view showing a pushing direction of a switch of a handpiece according to one example of a first modification of the first embodiment.

FIG. 9 is a plan view showing a pushing direction of a switch of a handpiece according to another example of the first modification of the first embodiment viewed from the opposite surface (top surface) direction.

FIG. 10 is a front view showing two second switches of a handpiece according to a second modification of the first embodiment.

FIG. 11 is a plan view showing a switch and a button viewed from the opposite surface (top surface) direction by cutting away part of the handle main body of the handpiece of the second embodiment.

FIG. 12 is a plan view showing a state in which the switch of the handpiece of the second embodiment is pushed with the thumb of the right hand.

FIG. 13 is a front view showing a handpiece of an energy treatment instrument of a third embodiment and showing a pushing direction of a switch by an arrow.

FIG. 14 is a plan view showing a handpiece and a switch of an energy treatment instrument of a fourth embodiment viewed from the opposite surface (top surface) and showing a pushing direction of the switch by an arrow.

### DETAILED DESCRIPTION

### First Embodiment

A first embodiment of an energy treatment instrument of the present invention will be described with reference to FIGS. 1 to 10.

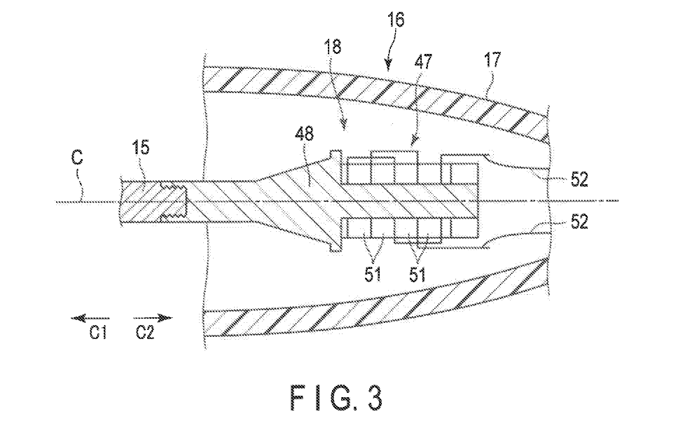

As illustrated in FIGS. 1 and 3, an energy treatment instrument 11 includes a handpiece 12, an electric power source unit 13, a cable 14 which connects the handpiece 12 and the electric power source unit 13, and a transducer unit 16 which supplies ultrasonic energy to a probe 15 of the handpiece 12. The transducer unit 16 includes a case 17 which is detachable from the handpiece 12 (handle main body 21), and a vibration generator 18 (transducer) which is stored in the case 17. In the present embodiment, one of two directions parallel to the longitudinal axis C of the probe 15 is referred to as a distal direction C1, and a direction opposite to the distal direction C1 is referred to as a proximal direction C2.

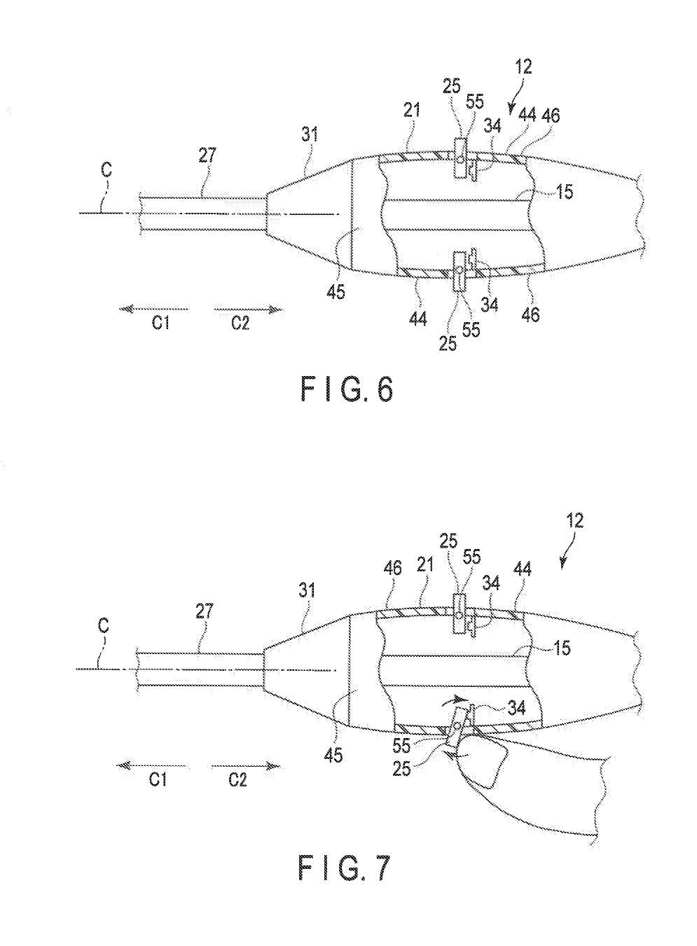

As illustrated in FIGS. 1 to 3, the handpiece 12 includes a handle main body 21 which constitutes part of an outer shell; a grip portion 22 which projects in a rod-shaped manner from the handle main body 21; a movable handle 24 which is rotatably attached to a finger-placement portion 23; a pair of switches 25 which are provided in the handle main body 21; a second switch 26 which is provided at the distal direction C side of the handle main body 21; a rod-shaped probe 15 (vibration transmission member) which is connected to the vibration generator 18; a cylindrical sheath 27 which is attached to the handle main body 21 in a manner to cover the periphery of the probe 15; an end effector 28 Which is provided at the distal end of the sheath a knob 31 which is fixed to the sheath 27; a jaw 32 which is provided to be rotatable relative to the probe 15 and the sheath 27; a cylindrical movable pipe 33 which is provided inside the sheath 27 and is advanced and retreated when the jaw 32 is opened and closed.; a button 34 (see FIG. 6) which is pushed by operation of the switch 25 in the handle main body 21; and a second button (not shown) which is pushed by operation of the second switch 26. The end effector 28 includes a treatment portion 35 at the distal end side of the probe 15, and the jaw 32. The probe 15 and the sheath 27 constitute a support portion 36. The end effector 28 is provided at the distal end of the support portion 36, and includes a portion directly abutting on tissue to be treated to perform various kinds of treatment on the tissue. The longitudinal axis of the support portion 36 coincides with the longitudinal axis C of the probe 15.

As illustrated in FIG. 1, the electric power source unit 13 includes an ultrasonic current supply section 41 (ultrasonic energy supply section), a high-frequency current supply section 42 (high-frequency energy supply section), and a controller 43 which controls them. The controller 43 can control supply of an ultrasonic wave generation current from-the ultrasonic current supply section 41 and supply of a high-frequency current from the high-frequency current supply section 42. When the operation of the switch 25 or the second switch 26 is detected by the button 34 and the second button, the controller 43 supplies the ultrasonic wave generation current from the ultrasonic current supply section 41 to the vibration generator 18, supplies the high-frequency current from the high-frequency current supply section 42 to the end effector 28, or performs both of them.

In the present embodiment, two types of energy, i.e., ultrasonic energy and high-frequency energy, are used as energy for treatment, but the combination of energy is not limited thereto. For treatment energy, besides the above, any one of ultrasonic energy, high-frequency energy, and thermal energy may be output alone, and these three types of energy may be output in combination in a suitable manner.

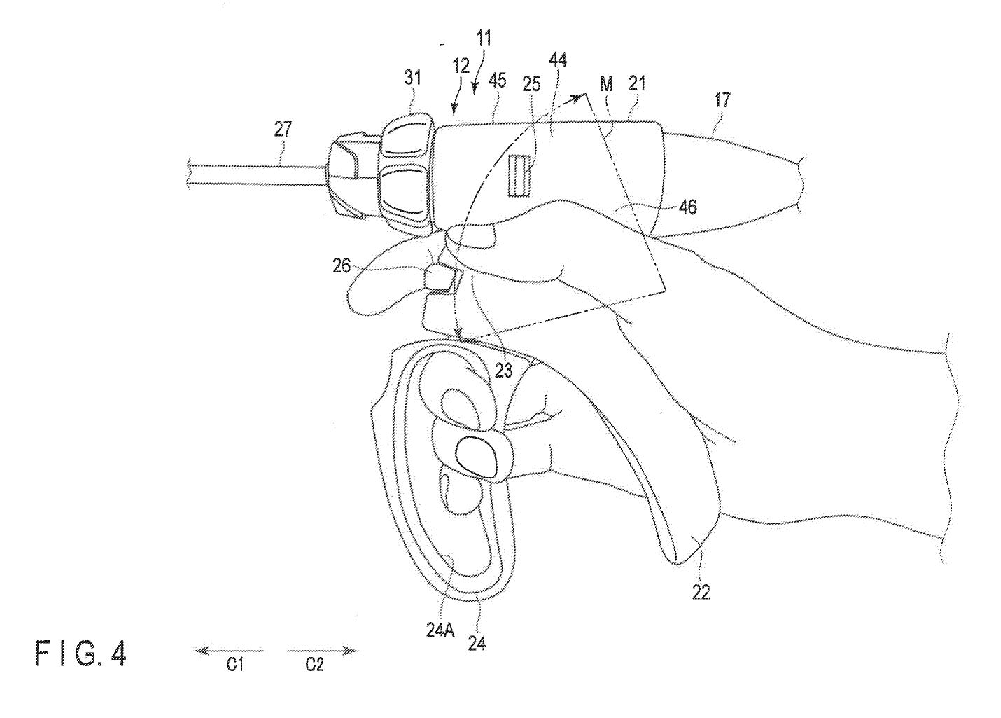

As illustrated in FIGS. 1 to 4, the handle main body 21 supports the support portion 36. The handle main body 21 includes a cylindrical portion 44 having an approximately cylindrical shape to surround the periphery of the probe 15, and a finger-placement portion 23 (neck portion) provided integrally with the cylindrical portion 44. The finger-placement portion 23 is a base position of the handle main body 21, and constitutes a portion in which an index finger and a thumb are placed when gripped by a doctor (see FIG. 4). The handle main body 21 includes an opposite surface 45 (top surface) positioned opposite to the grip portion 22, and a pair of side surfaces 46 positioned between the opposite surface 45 anal the grip portion 22. The side surfaces 46 stride over both sides of the cylindrical portion 44 and the finger-placement portion 23. The opposite surface 45 is constituted by a curved surface along the outer periphery of the cylindrical portion 44, but may have any surface shape as long as it is positioned on the side opposite to the grip portion 22. A surface may be flat or uneven, for example.

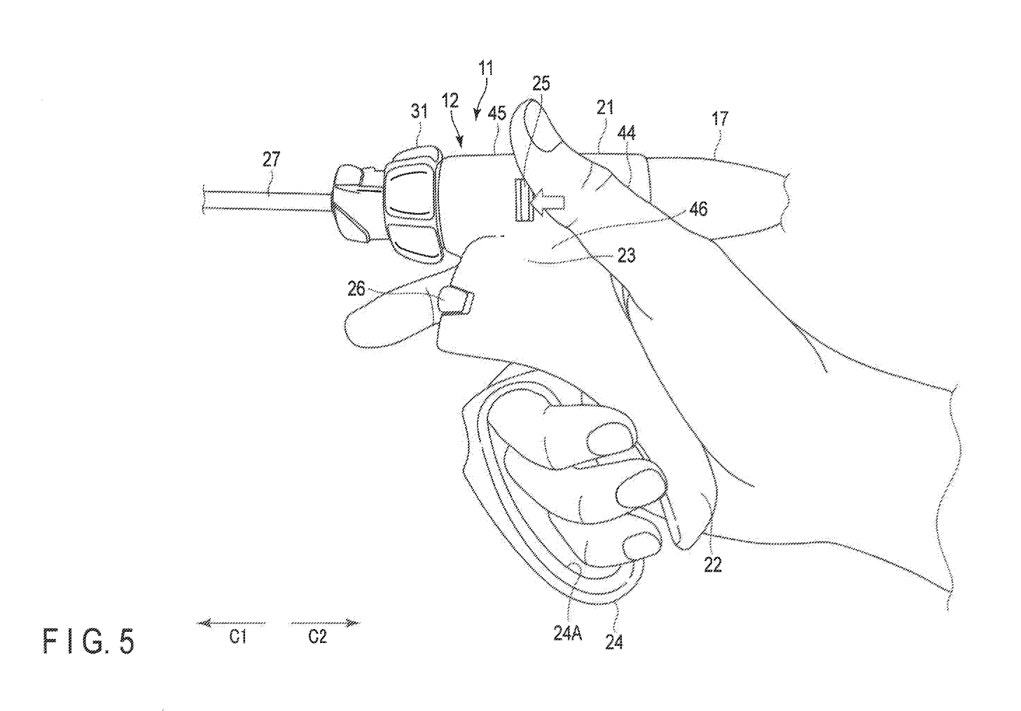

The movable handle 24 is attached to be movable (rotatable) relative to the finger-placement portion 23 of the handle main body 21. By bringing the movable handle 24 closer to or away from the grip portion 22, the doctor advances and retreats the movable pipe 33 inside the sheath 27, thereby enabling the end effector 28 to be opened and closed. Specifically, as illustrated in FIG. 5, by bringing the movable handle 24 closer to the grip portion 22, the jaw 32 abuts on the treatment portion 35 of the probe 15 (that is, performing a closing operation). On the other hand, as illustrated in FIG. 4, by moving the movable handle 24 away from the grip portion 22, the jaw 32 moves away from the treatment portion 35 of the probe 15 (that is performing an opening operation).

As shown in FIG. 3, the vibration generator 18 includes an ultrasonic transducer 47 and a horn member 48. The ultrasonic transducer 47 includes a plurality of piezoelectric elements 51 (four piezoelectric elements in the present embodiment) for changing a current into ultrasonic vibration. The ultrasonic transducer 47 is connected to one end of a first electric line 52. The first electrical line 52 extends inside the cable 14 and connects with an ultrasonic current supply section 41 of the electronic power supply unit 13 at the other end. When power is supplied from the ultrasonic current supply section 41 to the ultrasonic transducer 47 through the first electrical line 52, the ultrasonic transducer 47 generates ultrasonic vibrations.

As shown in FIG. 3, the ultrasonic transducer 47 is attached to the horn member 48. The horn member 48 is made of a metallic material. The horn member 48 is provided with an approximately cone-shaped cross-section transition portion, whose cross-sectional area decreases in the distal direction C1 of the probe 15. The amplitude of the ultrasonic vibration, which is generated in the ultrasonic transducer 47, is increased in the cross-sectional area transition portion.

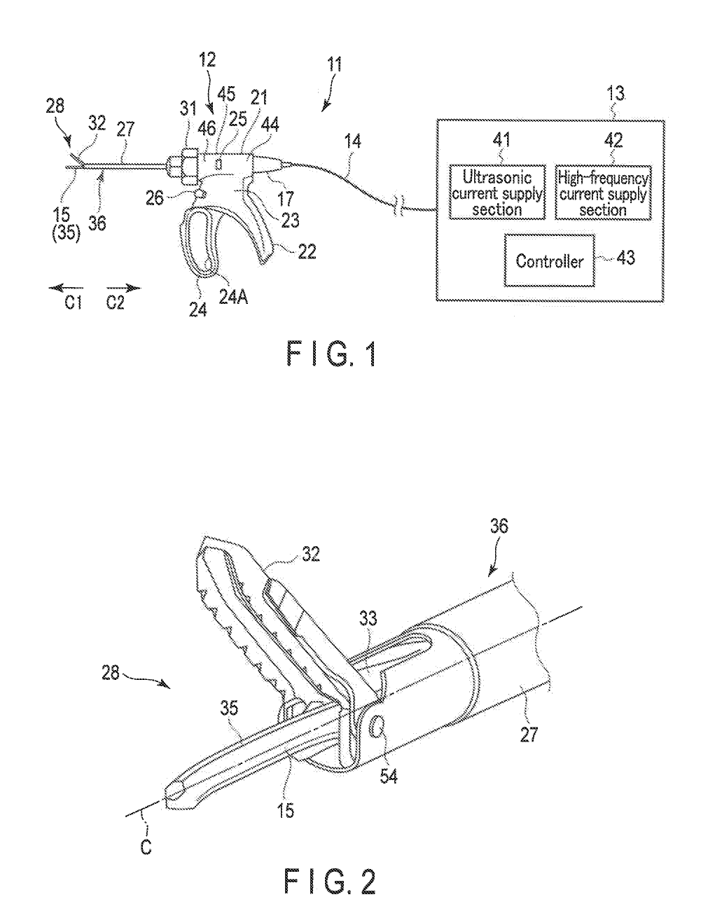

The probe 15 is formed of, for example, a biocompatible metallic material (e.g. a titanium alloy, etc.) in a rod shape. As illustrated in FIGS. 1 and 2, the proximal direction C2 side of the probe 15 is supported by the handle main body 21. A treatment portion 35 (blade) constituting a portion that comes in contact with the living tissue is provided at the distal direction C1 side of the probe 15. The proximal direction C2 side of the probe 15 is connected to one of two second electric lines. This one of the second electric lines extends inside the cable 14 and connects with one output terminal of a high-frequency supply section 42 at the other end.

That is, to the probe 15, ultrasonic vibration is transmitted from the vibration generator 18, and a high-frequency current is supplied from the high-frequency current supply section 42. Therefore, the probe 15 not only applies ultrasonic vibration to living tissues but functions as one pole of a bipolar electrode (end effector 28) for performing bipolar treatment.

The sheath 27 has a cylindrical shape, and protects the probe 15 located inside. The proximal direction C2 side of the sheath 27 is attached to the handle main body 21 to be rotatable with respect to the handle main body 21. The knob 31 is fixed to the sheath 27, and is attached to be rotatable with respect to the handle main body 21. By rotating the knob 31 with respect to the handle main body 21, the sheath 27, the probe 15, the ultrasonic transducer 47, and the jaw 32 can be integrally rotated about the longitudinal axis C. The sheath 27 includes a support pin 54 for supporting the jaw 32 at the distal end. The proximal end of the sheath 27 is connected to the other one of two second electric lines. The other one of the second electric lines extends inside the cable 14 and connects with the other output terminal of the high-frequency current supply section 42 at the other end.

The jaw 32 is rotatable about the support pin 54 between a contact position where the jaw 32 is brought into contact with the probe 15, and a spaced position where the jaw 27 is spaced apart from the probe 15. The jaw 32 is electrically connected to the sheath 27 via the support pin 54. Thus, the jaw 32 at the distal end of the sheath 27 functions as the other pole of the bipolar electrode (end effector 28) for performing bipolar treatment. The electrode part of the jaw 32 is made of, for example, copper alloy and the like.

One switch 25 is provided on one side surface 46 of the handle main body 21, and one switch 25 is provided on the other side surface 46. Therefore, the switches 25 are provided on both sides of the handle main body 21 in pairs. The pair of switches 25 (the switch and the third switch) is symmetrical to the center portion of the handle main body 21. As illustrated in FIG. 4, in a posture which the doctor holds the handle main body 21 with the right hand and holds the handle main body 21 (the finger-placement portion 23) between the base position of the thumb and the base position o the index finger, the pair of switches 25 is provided at a natural position within a movable range M of the thumb schematically shown by a two-dot chain line in FIG. 4. More specifically, the pair of switches 25 is provided at positions deviated from the finger-placement portion 23, that is, provided in the cylindrical portion 44.

As illustrated in FIG. 6, the switch 25 has a so-called seesaw shape which is rotatably supported by a shaft member 55 provided inside the handle main body 21. As illustrated in FIG: 7, the pushing direction (operation direction) of the switch 25 is a direction substantially along the longitudinal axis C of the probe 15 and the sheath 27. The switch 25 is pushed toward the distal side from the proximal side in a direction along the longitudinal axis C. The button 34 for detecting the pushing of the switch 25 is provided on the first switch board.

The switch 25, for example, corresponds to outputs of both high-frequency energy and ultrasonic energy, and corresponds to a coagulation/incision mode in which living tissue is incised while coagulation/blood stanching is carried out. That is, while pushing the switch 25, the doctor can turn on the output of high-frequency energy and ultrasonic energy to the treatment target (living tissue) from the end effector 28, and can turn off the output of high-frequency energy and ultrasonic energy by releasing the pushing of the switch 25. That is, by pushing the switch 25 or releasing the pushing, it is possible to change an ON/OFF status of the energy output from the end effector 28.

As illustrated in FIGS. 1 and 4, the second switch 26 is provided on a surface at the distal direction C1 side (distal end surface) in the longitudinal axial C direction of the handle main body 21 (finger-placement portion 23). The second switch 26 is pushed toward the proximal side from the distal side in the longitudinal axis C direction of the support portion 36. The second button for detecting the pushing of the second switch 26 is provided on a second switch board (not shown). The second switch 26, for example, corresponds to the output of ultrasonic energy, and corresponds to the incision mode for mainly incising the living tissue. That is, while pushing the second switch 26, the doctor can turn on the output of ultrasonic energy to the treatment target (living tissue) from the end effector 28 (treatment portion 35), and can turn off the output of ultrasonic energy by releasing the pushing of the switch 25. Namely, by pushing the second switch 26 or releasing the pushing, it is possible to change an ON/OFF status of the energy output from the end effector 28.

The energy output corresponding to the switch 25 and the second switch 26 is only an example. The switch 25 may correspond to the output of ultrasonic energy, and the second switch 26 may correspond to the outputs of both high frequency energy and ultrasonic energy.

Next, an operation of the energy treatment instrument 11 of the present embodiment will be described with reference to FIG. 4 to FIG. 7. For example, the doctor can hold the handpiece 12 of the energy treatment instrument 11 with the right hand (which may be the left hand) in a posture as illustrated in FIG. 4. At this time, the thumb and the index finger of the right hand, for example, are placed on the finger-placement portion 23. The middle finger, the ring finger, and the little finger of the right hand are passed through a ring portion 24A of the movable handle 24.

As shown in FIG. 5, the doctor pulls the movable handle 24 toward the grip portion 22 side with the middle finger, the ring finger, and the little finger of the right hand (or the left hand), thereby rotating the jaw 32 and bringing the jaw 32 in contact with the treatment portion 35 of the probe 15. When the living tissue is present between the jaw 32 and the treatment portion 35, it is possible to hold the living tissue between the treatment portion 35 and the jaw 32 like a forceps.

Furthermore, in a state where the living tissue is held as described above, when the switch 25 is pushed toward the distal side from the proximal side in the longitudinal axis C direction with the thumb of the right hand (or the left hand) as shown in FIG. 5, the switch 25 rotates around the shaft member 55 as shown in FIG. 6 and FIG. 7, and the button 34 is pushed at the portion located in the handle main body 21 of the switch 25. As a result, the button 34 detects an input of an ON/OFF status change of the energy output. While the button 34 is pushed, the controller 43 controls the ultrasonic current supply section 41 and the high-frequency current supply section 42 to turn on the output of ultrasonic energy and high-frequency energy from the end effector 28 (treatment portion 35). When the doctor releases the pushing of the switch 25, the switch 25 is returned to its original position by a spring (not shown) (for example, a torsion coil spring, or the like). Thereby, the controller 43 turns off the output of ultrasonic energy and high-frequency energy.

The switch 25 is provided in the movable range M of the thumb of the right hand (or the left hand). Thus, the doctor can push the switch 25 in a natural posture without difficulty. The doctor can push the switch 25 with the finger-placement portion 23 being held between the base position of the thumb and the base position of the index finger of the right hand (or the left hand). Therefore, the treatment portion 35 and the end effector 28 at the distal end side do not deviate, and operability for the doctor is excellent.

Similarly, when the second switch 26 is pushed by the doctor, the second button 34 detects an input of an ON/OFF status change of the energy output. The controller 43 controls the ultrasonic current supply section 41 to output the ultrasonic energy from the end effector 28 (treatment portion 35).

According to the first embodiment, the energy treatment instrument 11 includes: an end effector 28 provided at a distal end of a support portion 36; a handle main body 21 supporting the support portion 36; movable handle 24 attached to be movable with respect the handle main body 21 for operating the end effector 28; and a switch 25 provided on aside surface 46 of the handle main body 21, and capable of changing an ON/OFF status of an energy output from the end effector 28 when operated in a direction substantially along a longitudinal axis of the support portion 36.

According to this configuration, since the switch 25 is operated in the direction along the longitudinal axis of the support portion 36, the doctor can have an operational feeling comparable to the conventional trigger-type switch 25. Furthermore, since the switch 25 is provided on the side surface 46 of the handle main body 21, the switch 25 can be arranged at a position which is not conventionally known. Moreover, since the switch 25 is operable in the direction along the longitudinal axis C and provided on the side surface 46 of the handle main body 21, operation with the thumb is better for the doctor in terms of operability. Thus, the switch 25 can be operated using the thumb which can exert a greater force than other fingers, and thus the burden on the doctor's fingers can be reduced. This can reduce the burden on the doctor in a long operation.

The energy treatment instrument 11 is provided at the distal side in the longitudinal axis C direction of the handle main body 21, and includes the second switch 26 capable of changing an ON/OFF status of the energy output from the treatment portion 35. According to this configuration, the so-called trigger-type second switch 26 can be provided independent from the switch 25 on the side surface 46 of the handle main body 21. Therefore, the fingers can be used separately in such a manner that the thumb operates the switch 25 and the index finger operates the switch 26. This reduces the risk that the doctor erroneously operates the switches 25 and 26, thereby improving the safety of the operation and the convenience for the doctor.

The energy treatment instrument 11 includes a shaft member 55 which rotatably supports the switch 25, and a button 34 which is provided inside the handle main body 21 and pushed by the switch 25 rotating about the shaft member 55 to detect an input of an ON/OFF status change of the energy output.

According to this configuration, since the switch 25 can be a so-called seesaw form, it is possible to reduce a space required for providing the switch 25 as compared to a slide-type switch, and also to reduce the length of stroke required for operating the switch 25.

In this case, the handle main body 21 includes an opposite surface 45 located opposite to the side on which the movable handle 24 is provided, and the side surface 46 is located between the movable handle 24 and the opposite surface 45. According to this configuration, it is possible to provide the switch 25 on the side surface 46 including a portion in which the fingers other than the fingers holding the movable handle 24 are placed, and the doctor can operate the switch 25 with the handle main body 21 being held in a natural manner. As a result, the operability of the energy treatment instrument 11 can be improved.

The switch 25 is located within the movable range M of the thumb in a posture in which the handle main body 21 is held between the base position of the thumb and the base position of the index finger. According to this configuration, the doctor can operate the switch 25 in a comfortable posture using the thumb with the handle main body 21 being held. Furthermore, since the switch 25 can be operated with the handle main body 21 being held between the base position of the thumb and the base position of the index finger, it is possible to reduce the risk that the end effector 28 deviates when the switch 25 is operated.

The handle main body 21 includes the finger-placement portion 23 in which fingers are placed when gripped by hand, and the switch 25 is provided at a position deviated from the finger-placement portion 23. According to this configuration, the switch 25 can be disposed at a position deviated from the finger-placement portion 23, and it is possible to prevent the handle main body 21 from being difficult to hold due to the switch 25 provided.

The function corresponding to the switch 25 is different from the function corresponding to the second switch 26. According to this configuration, since the switch 25 and the second switch 26 provided at different positions can have different functions, erroneous operation by the doctor can be prevented.

The switch 25 is operated toward the distal side from the proximal side of the support portion 36 with respect to the direction along the longitudinal axis C direction. According to this configuration, the operation direction of the switch 25 can be a direction in which operation can be easily performed with the thumb in terms of ergonomics. This further improves the operability of the energy treatment instrument 11.

The energy treatment instrument 11 includes a third switch 25 provided on a side opposite to the switch 25 with respect to the central portion of the handle main body 21. According to this configuration, it is possible to dispose the switch 25 or the third switch 25 at a position where operation can be performed with the thumb regardless of whether the handle main body 21 is held with the right hand or the left hand. Since it can be used in both cases where the doctor uses the instrument with the right hand and uses it with the left hand, the energy treatment instrument 11 with high versatility can be provided.

In the first embodiment described above, the pushing direction (operating direction) of the switch 25 (the switch and the third switch) is the direction along the longitudinal axis C, but the operating direction of the switch 25 is not limited thereto. The pushing direction (operating direction) of the switch 25 only has to be a direction substantially along the longitudinal axis C.

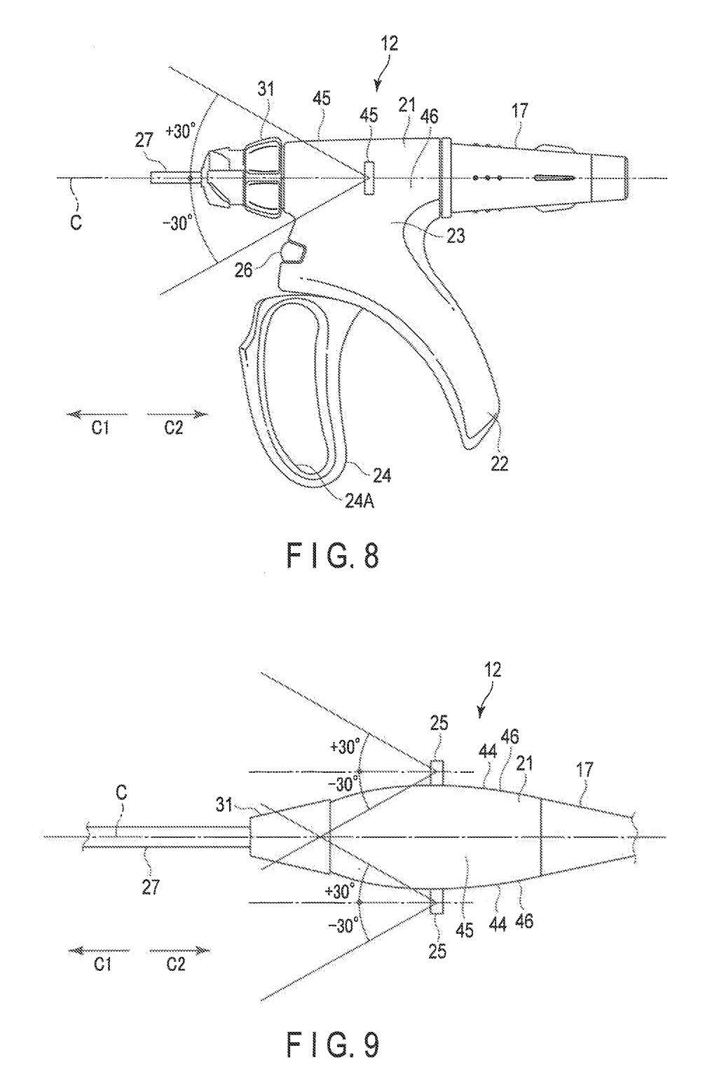

In the modifications described below, the parts different from those of the first embodiment will he described, and explanation of the parts identical to those of the first embodiment will be omitted. In the first modification of the first embodiment, for example, the pushing direction (operating direction) of the switch 25 may be a direction inclined in a range of 30° or less from the longitudinal axis C. In this case, the pushing direction of the switch 25 can be, for example, oblique to a direction away from the probe 15 (end effector 28) toward the distal direction C1. More specifically, the pushing direction of the switch 25 may be inclined by an angle of ±30° or less from the longitudinal axis C when the side surface 46 is viewed as a front view as illustrated in FIG. 8, or may be inclined by an angle of ±30° or less from the longitudinal axis C when the opposite surface 45 (top surface) is viewed as a front view as illustrated in FIG.

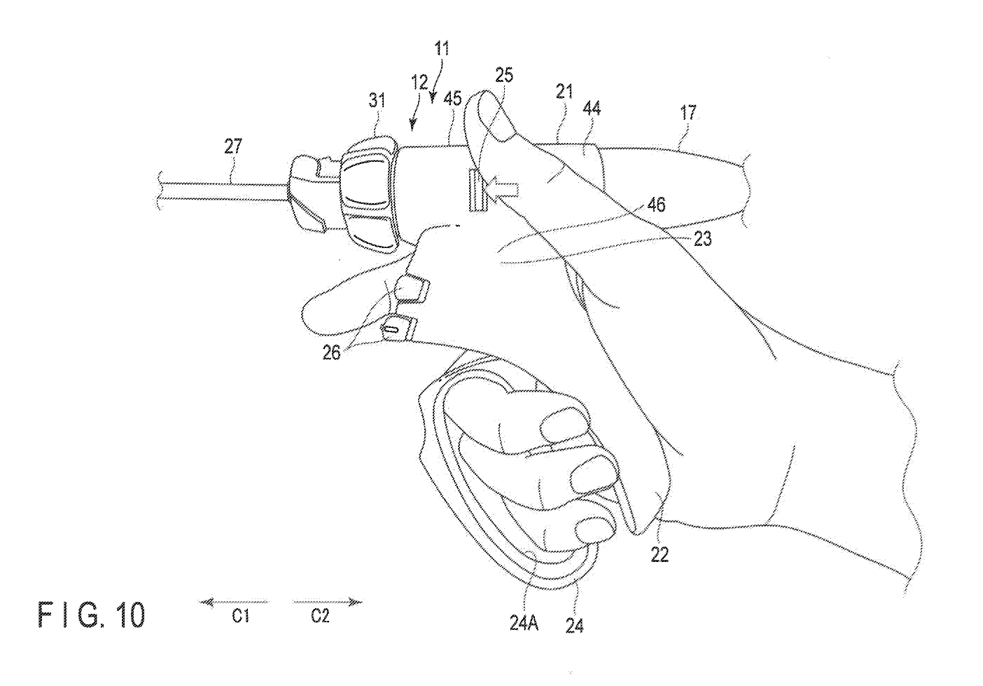

In the present embodiment, one second switch 26 is provided, but the number of the second switches 26 is not limited to one. If space allows, two second switches 26 may be provided in parallel as in the second modification of the first embodiment shown in FIG. 10. In this case, for example, the switch 25 corresponds to the treatment (function) of the coagulation/incision mode for outputting both high-frequency energy and ultrasonic energy as in the first embodiment. One of the second switches 26 (the side close to the movable handle 14) corresponds to, for example, the treatment (function) of the incision mode for outputting ultrasonic energy to mainly incise living tissue. The other side of the second switches 26 (the side close to the sheath 27) may be configured to correspond to, for example, the treatment (function) of the coagulation mode for outputting high-frequency energy to perform coagulation/blood stanching of the living tissue. In this modification, each function of the switch 25 and the two second switches 26 is assigned in a discretionary manner. Furthermore, the function corresponding to each switch 25 may be set to be changeable in a discretionary manner. In this case, for example, the function corresponding to each switch 25 may be set to be changeable by the doctor's operation of the electric power source unit 13.

Furthermore, the combination of energy types corresponding to each switch 25 is not limited to the above. When the energy treatment instrument 11 can output, for example, thermal energy other than ultrasonic energy and high-frequency energy, a function corresponding to an output of a suitable combination of ultrasonic energy, high-frequency energy, and thermal energy described above may be assigned to each switch.

### Second Embodiment

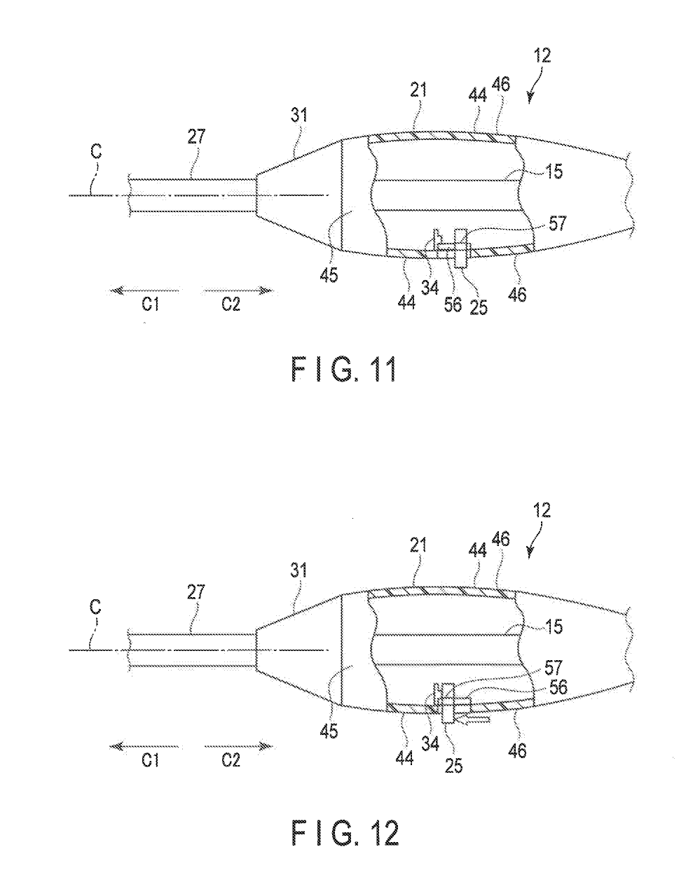

An energy treatment instrument 11 according to the second embodiment will be described with reference FIG. 11 and FIG. 12. The second to fourth embodiments described below include parts common to the first embodiment. Therefore, in each embodiment below, mainly the parts different from those of the first embodiment will be explained, and illustrations or explanations of the parts identical to those of the first embodiment will be omitted.

The switch 25 is configured to be slidable along the longitudinal axis C. Specifically, a groove 56 that extends in a direction along the longitudinal axis C is formed in the handle main body 21. At the switch 25 side, a quadrangular prism-shaped pin 57 projects so as to fit into the groove 56. As the pin 57 slides relative to the groove 56, the switch 25 is slidable in the direction along the longitudinal axis C. Although not shown in the figures, the switches 25 are provided in pairs as in the first embodiment, and the pair of switches 25 (the switch and the third switch) is symmetrical with respect to the center portion of the handle main body 21. The button 34 is provided at a position overlapping the switch 25 with respect to the longitudinal axis C. The pair of switches 25 is provided at a natural position within a movable range M of the thumb when the doctor holds the handle main body 21 with the right hand (or the left hand) in the holding manner shown in FIG. 4.

Next, an operation of the energy treatment instrument 11 of the present embodiment will be described with reference to FIG. 11 and FIG. 12. For example, in the same manner as the first embodiment, the doctor can hold the handpiece 12 of the energy treatment instrument 11 with the right hand (or the left hand) in a posture as shown in FIG. 4. As shown in FIG. 5, the doctor can hold the living tissue between the treatment portion 35 and the jaw 32 like a forceps by pulling the movable handle 24 to the grip portion 22 side.

When the doctor pushes the switch 25 with the thumb of the right hand (or the left hand) toward the distal side from the proximal side of the longitudinal axis C with the living tissue being held between the jaw 32 and the treatment portion 35, the switch 25 slides in the longitudinal axis C direction as shown in FIG. 11 and FIG. 12, and the button 34 is pushed at a portion located in the handle main body 21 of the switch 25. Thus, while the switch 25 is pushed, the output of ultrasonic energy and high-frequency energy from the end effector 28 (treatment portion 35) is turned on. When the doctor releases the pushing of the switch 25, the switch 25 is returned to its original position by a spring (not shown) (for example, a tension coil spring or the like), and the output of ultrasonic energy and high frequency energy is turned off.

Since the switch 25 is provided within the movable range M of the thumb of the right hand, the doctor can push the switch 25 without difficulty with a natural posture. Furthermore, the doctor can push the switch 25 with the finger-placement portion 23 being held between the base position of the thumb and the base position of the index finger of the right hand (or the left hand). Therefore, the treatment portion 35 and the end effector 28 at the distal end side do not deviate, and operability for the doctor is excellent.

According to the present embodiment, the button 34 is provided at a position overlapping the switch 25 with respect to the longitudinal axis C direction. According to this configuration, since the switch 25 can be of a so-called slide type, the length of the stroke of the switch 25 can be sufficiently secured. Thereby, even when the doctor erroneously touches the switch 25, energy is not output from the end effector 28 (treatment portion 35), and it is possible to provide the energy treatment instrument 11 with improved safety.

In the second embodiment, as in the first modification of the first embodiment, the pushing direction of the switch 25 may be inclined by an angle of ±30° or less with respect to the longitudinal axis C, or as in the second modification of the first embodiment, a plurality of second switches 26 may be provided.

### Third Embodiment

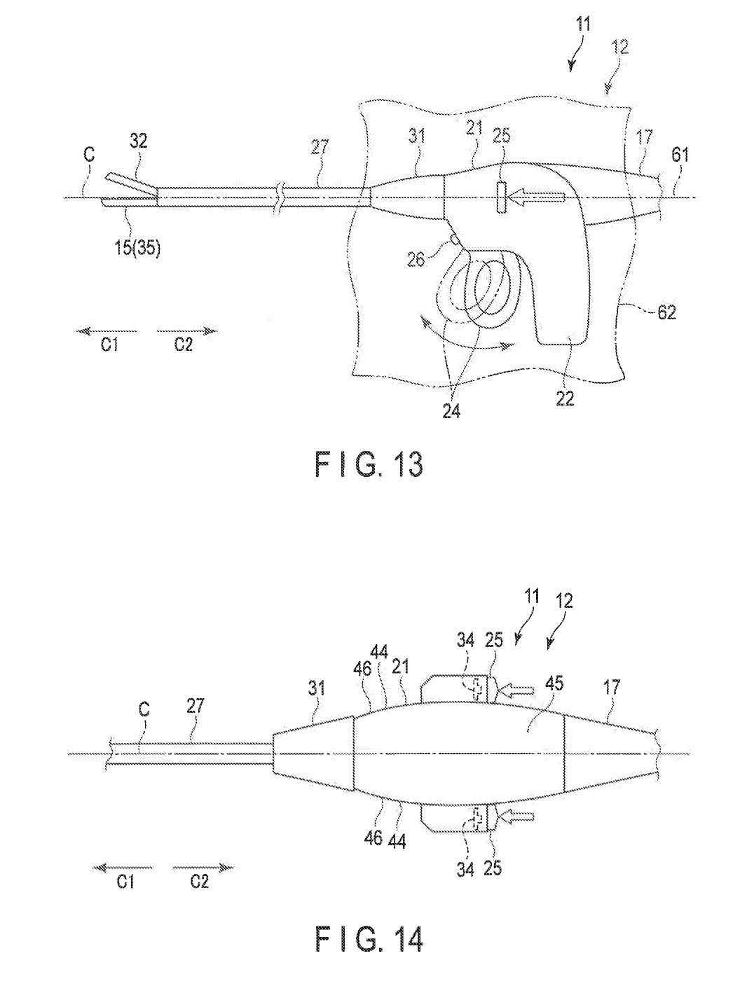

An energy treatment instrument 11 according to the third embodiment will be described with reference to FIG. 13. The switch 25 is provided on a plane 51 including a longitudinal axis C of the probe 15 (support portion 36) and orthogonal to a plane 62 that is formed by locus of the rotating movable handle 24. The shape of the switch 25 and the structure for supporting the switch 25 are the same as those of the first and second embodiments.

Next, an operation of the energy treatment instrument 11 of the present embodiment will be described with reference to FIG. 13. As in the first embodiment, the doctor can hold the handpiece 12 of the energy treatment instrument 11 with the right hand (or the left hand) in a posture as shown in FIG. 4. The doctor can hold the living tissue between the treatment portion 35 and the jaw 32 like a forceps by pulling the movable handle 24 to the grip portion 22 side as shown in FIG. 5.

Furthermore, when the doctor pushes the switch 25 toward the distal side from the proximal side in the longitudinal axis C direction with the thumb of the right hand (or the left hand) with the living tissue being held as described above, the switch 25 rotates around the shaft (shaft member 55) and the button 34 is pushed at the portion located in the handle main body 21 of the switch 25. Thus, the output of ultrasonic energy and high-frequency energy from the end effector 28 (treatment portion 35) is turned on.

Since the switch 25 is provided within the movable range M of the thumb of the right hand, the doctor can push the switch 25 without difficulty with a natural posture. The doctor can push the switch 25 with the finger-placement portion 23 being held between the base position of the thumb and the base position of the index finger of the right hand (or the left hand). Therefore, the end effector 28 at the distal end side does not deviate, and operability for the doctor is excellent. Furthermore, in the present embodiment, since the switch 25 is located on the plane 61 including the longitudinal axis C of the probe 15, the component of the moment produced in the handle main body 21 and the end effector 28 caused by the pushing force of the switch 25 is only in the plane.

According to the third embodiment, the switch 25 is provided on the plane 61 including the longitudinal axis C and orthogonal to the plane 62 that is formed by locus of the moving movable handle 24. According to this configuration, the component of the moment applied to the handle main body 21 and the treatment portion 35 when the switch 25 is operated is only in the plane (component in the horizontal plane). As a result, even if the switch 25 is pushed with a strong force and a rotational force is generated in the handle main body 21 and the end effector 28, the rotational force is only the component in the direction in which the end effector 28 shakes the head, and no rotational force is generated in a direction in which the end effector 20 bows. Therefore, the end effector 28 does not deviate to a direction not expected by the doctor, and the operability of the energy treatment instrument 11 can be improved.

In the third embodiment, as in the first modification of the first embodiment, the pushing direction of the switch 25 may be inclined by an angle of ±30° or less with respect to the longitudinal axis C, or as in the second modification of the first embodiment, a plurality of second switches 26 may be provided.

### Fourth Embodiment

An energy treatment instrument 11 according to the fourth embodiment will be described with reference to FIG. 14. One switch 25 is provided on each of the side surfaces 46 of the handle main body 21. Therefore, the switches 25 are provided on both sides of the handle main body 21 in pairs. The pair of switches 25 (the switch and the third switch) is symmetrical to the center portion of the handle main body 21. The pair of switches 25 is provided at a natural position within a movable range M of the thumb when the doctor holds the handle main body 21 with the right hand in the holding manner shown in FIG. 4.

The switch 25 is constituted by a push-type switch 25 supported by a spring (not shown) (for example, compression coil spring or the like). The pushing direction (operating direction) of the switch 25 is a direction substantially along the longitudinal axis C. The switch 25 may be disposed on a projection provided on the handle main body 21 as shown in FIG. 14. The button 34 is provided at a position overlapping the switch 25 with respect to the longitudinal axis C, that is, at a position facing the switch 25.

An operation of the energy treatment instrument 11 of the present embodiment will be described. As in the first embodiment, for example, the doctor can hold the handpiece 12 of the energy treatment instrument 11 with the right hand (or the left hand) in a posture as shown in FIG. 4. As shown in FIG. 5, the doctor can hold the living tissue between the treatment portion 35 and the jaw 32 like a forceps by pulling the movable handle 24 to the grip portion 22 side.

When the doctor pushes the switch 25 with the thumb of the right hand toward the distal side from the proximal side in the direction of the longitudinal axis C direction with the living tissue being held as described above, the button 34 facing the switch 25 is pushed. Thereby, the output of ultrasonic energy and high-frequency energy from the end effector 28 (treatment portion 35) is turned on. Also, when the doctor releases the pushing of the switch 25, the switch 25 is returned to its original position by a spring (not shown), and the energy output is turned off.

Since the switch 25 is provided within the movable range M of the thumb of the right hand, the doctor can push the switch 25 without difficulty with a natural posture. The doctor can push the switch 25 with the finger-placement portion 23 being held between the base position of the thumb and the base position of the index finger of the right hand (or the left hand). Therefore, the treatment portion 35 and the end effector 28 at the distal end side do not deviate, and operability for the doctor is excellent

According to the present embodiment, the button 34 is provided at a position overlapping the switch 25 with respect to the longitudinal axis C direction. According to this configuration, since the switch can be a so-called push type, it is possible to provide the energy treatment instrument 11 with simplified structure and reduced manufacturing costs.

In the fourth embodiment, as in the first modification of the first embodiment, the pushing direction of the switch 25 may be inclined by an angle of 30° or less with respect to the longitudinal axis C, or as in the second modification of the first embodiment, a plurality of second switches 26 may be provided.

The present invention is not limited to the above-described embodiments, and can be modified as appropriate in practice without departing from the gist of the invention. In addition, it is, of course, possible to combine the energy treatment systems 11 of each of the above embodiments to configure one energy treatment system.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An energy treatment instrument comprising:
an end effector for treating tissue;
a support portion extending along a longitudinal axis, including a distal end and a proximal end, and including a distal side attached to the end effector;
a handle main body provided at a proximal side of the support portion, and including a portion formed to surround the proximal end of the support portion; and
a switch provided on a side surface of the portion surrounding the support portion in the handle main body, turning on an energy output from the end effector by being pushed toward the distal side from the proximal side of the longitudinal axis, and turning off the energy output by being returned to an original position by releasing pushing.

2. The energy treatment instrument according to claim 1, further comprising a second switch provided on a surface at the distal side of the handle main body, turning on an output of second energy different from the energy by being pushed toward to the proximal side from the distal side of the longitudinal axis, and turning off the output of the second energy by releasing the pushing.

3. The energy treatment instrument according to claim 1, comprising:
a shaft member rotatably supporting the switch; and
a button provided inside the handle main body and pushed by the switch rotating about the shaft member to detect an input of an ON/OFF status change of the energy output.

4. The energy treatment instrument according to claim 1, comprising:
a button provided inside the handle main body and detecting an input of an ON/OFF status change of the energy output,
wherein the switch is slidable in a direction substantially along the longitudinal axial, and
the button is pushed by the slide movement of the switch and is capable of detecting the input.

5. The energy treatment instrument according to claim 1,
wherein the handle main body includes an opposite surface located on a side opposite to a side on which a movable handle is provided, and
the side surface is located between the movable handle and the opposite surface.

6. The energy treatment instrument according to claim 1, wherein the switch is positioned within a movable range of a thumb in a posture in which the handle main body is held between a base position of a thumb and a base position of an index finger.

7. The energy treatment instrument according to claim 6, wherein
the handle main body includes a finger-placement portion in which fingers are placed when gripped by hand, and
the switch is provided at a position deviated from the finger-placement portion.

8. The energy treatment instrument according to claim 2, wherein a function corresponding to the switch is different from a function corresponding to the second switch.

9. The energy treatment instrument according to claim 4, wherein the button is provided at a position overlapping the switch with respect to the longitudinal axis.

10. The energy treatment instrument according to claim 1, wherein the switch is operated toward a distal side from a proximal side of the support portion with respect to the direction substantially along the longitudinal axis.

11. The energy treatment instrument according to claim 1, further comprising a third switch provided on a side opposite to the switch with respect to a center portion of the handle main body.

12. The energy treatment instrument according to claim 1, wherein the switch is provided on a plane including the longitudinal axis and orthogonal to a plane formed by a locus of a moving movable handle.

13. The energy treatment instrument according to claim 1, wherein part of the handle main body provided to surround the support portion is a cylindrical portion having a cylindrical shape.

14. The energy treatment instrument according to claim 7, wherein the second switch is provided on the finger-placement portion.

15. The energy treatment instrument according to claim 1, further comprising a movable handle attached to be movable with respect to the handle main body for operating the end effector.
